(19) United States
(12) Patent Application Publication
(10) Pub. No.: US 2004/0197813 A1
(43) Pub. Date: Oct. 7, 2004

(54) Computer system for providing information about the risk of an atypical clinical event based upon genetic information

(75) Inventors: Mark A. Hoffman, Lee's Summit, MO (US); David P. McCallie JR., Stilwell, KS (US)

Correspondence Address: SHOOK, HARDY & BACON LLP, 2555 GRAND BLVD, KANSAS CITY,, MO, 64108, US

(73) Assignee: CERNER INNOVATION, INC.

(21) Appl. No.: 10/826,595

(22) Filed: Apr. 16, 2004

Related U.S. Application Data
(62) Division of application No. 09/981,248, filed on Oct. 16, 2001.
(60) Provisional application No. 60/285,263, filed on Apr. 20, 2001.

(51) Int. Cl. C12Q 1/68; G06F 19/00; G01N 33/48; G01N 33/50

(52) U.S. Cl. 435/6; 702/20

(57) ABSTRACT

A method in a computer system for preventing atypical clinical events related to information identified by DNA testing a person is provided. The method includes receiving clinical agent information. The method also includes determining if a gene is associated with the clinical agent information, and if so, obtaining a genetic test result value for the associated gene of the person. The method further includes comparing the genetic test result value to a list of polymorphism values associated with an atypical clinical event, and determining whether the genetic test result value correlates to a polymorphism value on the list, and if so, outputting information about the atypical clinical event associated with the polymorphism value.

### CROSS-REFERENCE TO RELATED APPLICATIONS

This application claims the benefit of priority to U.S. patent application Ser. No. 09/981,248, filed Oct. 16, 2001, which claims the benefit of priority to U.S. Provisional Application No. 60/285,263, filed Apr. 20, 2001.

### STATEMENT REGARDING FEDERALLY SPONSORED RESEARCH OR DEVELOPMENT

Not applicable.

### TECHNICAL FIELD

The present invention relates to a computer system and, more particularly, to a computer system for providing information about the risk of an atypical clinical event based upon genetic information.

### BACKGROUND OF THE INVENTION

In the past, numerous approaches have been taken to administer drugs and pharmaceuticals safely. These approaches have sought to avoid adverse drug reactions (ADRs) such as adverse drug-drug interactions and drug allergy reactions. Despite a growing amount of information regarding drug interactions, allergenicity, proper dosages, pharmacology, side effects and other information regarding drugs and pharmaceuticals, an unreasonable number of ADRs continue to occur. As reported by the Institute of Medicine, an estimated 106,000 deaths occurred in 1994 due to ADRs, and more than 2,000,000 hospitalized patients experienced serious, if not fatal, ADRs. Lazarou J. et al., Incidence of adverse drug reactions in hospitalized patients: a meta-analysis of prospective studies, J. Am. Med. Assn. 1998: 279: 1200-1205. While many of these reactions are attributable to procedural errors, a significant percentage of these reactions were due to inadequate or incomplete information regarding the likely response a particular patient will have to the drug. In addition to ADRs, some patients receive little or no benefit from certain drugs. These atypical responses can lead to prolonged suffering, extended hospital stays and other social and financial costs incurred until an effective drug is identified and administered.

Much of the individual variability in the response to drugs can be attributed to heredity, yet this genetic information has not been fully considered in drug administration decisions. Genetic information has not yet been adequately incorporated into the decision making process due to a limited understanding of the correlation between genetic traits and the ability to metabolize a particular drug, limited availability of effective and inexpensive tests to determine a patient's genetic traits, and the lack of an integrated system for effectively storing and processing the voluminous and often complex genetic information.

Slowly, some of these deficiencies are being overcome. In recent years, genetic information has become increasingly available through research efforts such as the Human Genome Project. The study of variability in drug response due to heredity, known as pharmacogenetics, has lead to the discovery and understanding of gene to drug relationships. In other words, information about the manner in which certain drugs interact with the products of genes in the human body has been documented. Scientists have uncovered and continue to uncover a number of correlations between drug responses (or phenotypes) and the specific genetic makeup (or genotype) of a patient. Many variations in genotype have been clearly associated with variable responses to drugs.

At this point, the genetic variability in the human response to drugs has been largely attributed to the variations in drug/metabolizing enzyme (DME) genes, DME receptors and drug transporter genes. In other words, the pharmacogenetic differences in individuals appear most frequently in the genes responsible for the transformation or metabolism of drugs. The amount of variation in the DME genes, also known as a polymorphism, often accounts for the deviation in the drug response from the typical, desired response. Information about the individual's genetic deviation from atypical genetic trait can be predictive of whether or not the drug will be either toxic or inefficient at the recommended dosage. This information should be considered to avoid adverse, or other atypical, reactions. For example, genetic mutations can lead to DMEs that are either overactive, inactive or only moderately active. Typically, overactive DMEs require additional dosages of the drug or administration of an alternative drug. Inactive DMEs lead to an accumulation of the drug and drug toxicity, and moderately active DMEs require smaller dosages of the drug. Not only have the associations between a patient's genetic traits and the likely drug response been discovered and documented, but advances have been made to allow for affordable genetic testing of a specific patient for a relevant genetic mutation or mutations. As the relationships between individual mutations and drug reactions become increasingly known, and the costs of testing for these mutations drops, it is likely that the clinician's standard of care will soon require testing and consideration of a patient's genetic predisposition before administering drugs and pharmaceuticals to the patient.

However, as yet, this important information has not been integrated into an effective clinical process for managing and processing genetic information in an efficient manner. The complexity and volume of genetic information create challenges that have yet been met. A comprehensive system for considering preexisting and unchanging genetic traits in the decision making process has not been developed. Likewise, a system for considering a patient's demographic information in order to anticipate a likely genetic predisposition has not been employed. Moreover, an efficient system for referencing data structures that contain content relevant to the relationships between atypical reactions and drugs, and the likely risks associated with certain genetic mutations, has not been developed.

Accordingly, there is a need for an effective system and method for incorporating a patient's genetic information, either anticipated or determined by genetic testing, into the clinical decision making process. A need also exists for a system for processing genetic information that is integrated with a comprehensive healthcare system and is capable of providing information to the patient and triggering any of a variety of clinical actions within the construct of the healthcare system. Still another need is for a system that processes genetic data in a reliable and cost efficient manner to improve patient safety, reduce liability and produce efficiencies not previously realized. There is yet another need for a system and method that accesses information regarding newly discovered genetic associations and risks in an efficient manner. Still another need is for a system and method for providing information regarding agents that are affected by the products of specific genetic mutations.

### SUMMARY OF THE INVENTION

Generally described, a method in a computer system for preventing atypical clinical events related to information identified by DNA testing a person is provided. The method includes receiving clinical agent information. The method also includes determining if a gene is associated with the clinical agent information, and if so, obtaining a genetic test result value for the associated gene of the person. The method further includes comparing the genetic test result value to a list of polymorphism values associated with an atypical clinical event, and determining whether the genetic test result value correlates to a polymorphism value on the list, and if so, outputting information about the atypical clinical event associated with the polymorphism value.

In another aspect of the invention, a method in a computer system for preventing atypical clinical events related to information identified by DNA testing a person is provided. The method includes receiving clinical agent information and determining if a gene is associated with the clinical agent information. The method further includes inquiring if the person has a genetic test result value for the gene, and if not, generating an output including information regarding the likelihood that the person has a gene variant of the gene indicative of an atypical clinical event.

In yet another aspect of the invention, a method in a computer system for processing hereditary data related to the use of clinical agents by a person is provided. The method includes receiving a genetic test result value for the person. The method also includes determining if the genetic test result value is a polymorphism value associated with an atypical clinical event, and if so, accessing a list of risk-associated agents. The method further includes outputting an interpretation of the genetic test result value and the list of risk-associated agents.

Additional advantages and novel features of the invention will be set forth in part in a description which follows, and in part will become apparent to those skilled in the art upon examination of the following, or may be learned by practice of the invention.

### BRIEF DESCRIPTION OF THE SEVERAL VIEWS OF THE DRAWINGS

The present invention is described in detail below with reference to the attached drawing figures, wherein:

FIG. 1 is a schematic diagram of a suitable computing system environment for use in implementing the present invention;

FIG. 2 is a flow diagram illustrating a preferred method for providing information of genetically attributable risks associated with a specific agent;

FIG. 3 illustrates an agent selection window;

FIG. 4 illustrates a genetic test ordering window;

FIG. 5 illustrates a notification window; and

FIG. 6 is a flow diagram illustrating a preferred method of providing information of genetically attributable risks associated with a genetic test result value.

### DETAILED DESCRIPTION OF THE INVENTION

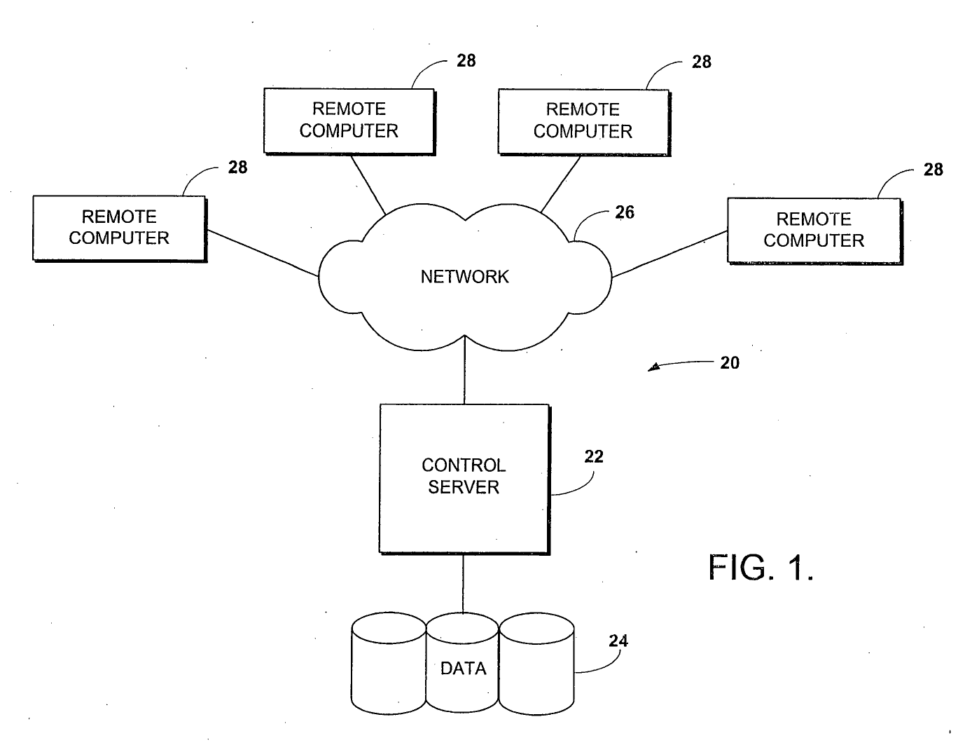

The present invention provides a method and system providing information about the risk of an atypical clinical event based upon genetic information. FIG. 1 illustrates an example of a suitable medical information computing system environment 20 on which the invention may be implemented. The medical information computing system environment 20 is only one example of a suitable computing environment and is not intended to suggest any limitation as to the scope of use or functionality of the invention. Neither should the computing environment 20 be interpreted as having any dependency or requirement relating to any one or combination of components illustrated in the exemplary environment 20.

The invention is operational with numerous other general purpose or special purpose computing system environments or configurations. Examples of well-known computing systems, environments, and/or configurations that may be suitable for use with the invention include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The invention may be described in the general context of computer-executable instructions, such as program modules, being executed by a computer. Generally, program modules include routines, programs, objects, components, data structures, etc. that perform particular tasks or implement particular abstract data types. The invention may also be practiced in distributed computing environments where tasks are performed by remote processing devices that are linked through a communications network. In a distributed computing environment, program modules may be located in both local and remote computer storage media, including memory storage devices.

With reference to FIG. 1, an exemplary medical information system for implementing the invention includes a general purpose computing device in the form of server 22. Components of server 22 may include, but are not limited to, a processing unit, internal system memory, and a suitable system bus for coupling various system components, including database cluster 24 to the control server 22. The system bus may be any of several types of bus structures, including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronic Standards Association (VESA) local bus, and Peripheral Component Interconnect (PCI) bus, also known as Mezzanine bus.

Server 22 typically includes therein or has access to a variety of computer readable media, for instance, database cluster 24. Computer readable media can be any available media that can be accessed by server 22, and includes both volatile and nonvolatile media, removable and nonremovable media. By way of example, and not limitation, computer readable media may comprise computer storage media and communication media. Computer storage media includes both volatile and nonvolatile, removable and nonremovable media implemented in any method or technology for storage of information, such as computer readable instructions, data structures, program modules or other data. Computer storage media includes, but is not limited to, RAM, ROM, EEPROM, flash memory or other memory technology, CD-ROM, digital versatile disks (DVD), or other optical disk storage, magnetic cassettes, magnetic tape, magnetic disk storage, or other magnetic storage devices, or any other medium which can be used to store the desired information and which can be accessed by server 22. Communication media typically embodies computer readable instructions, data structures, program modules, or other data in a modulated data signal, such as a carrier wave or other transport mechanism, and includes any information delivery media. The term “modulated data signal” means a signal that has one or more of its characteristics set or changed in such a manner as to encode information in the signal. By way of example, and not limitation, communication media includes wired media, such as a wired network or direct-wired connection, and wireless media such as acoustic, RF, infrared and other wireless media. Combinations of any of the above should also be included within the scope of computer readable media.

The computer storage media, including database cluster 24, discussed above and illustrated in FIG. 1, provide a storage of computer readable instructions, data structures, program modules, and other data for server 22.

Server 22 may operate in a computer network 26 using logical connections to one or more remote computers 28. Remote computers 28 can be located at a variety of locations in a medical environment, for example, but not limited to, hospitals, other inpatient settings, pharmacies, a clinician's office, ambulatory settings, testing labs, medical billing and financial offices, hospital administration, and a patient's home environment. Clinicians include, but are not limited to, the treating physician, specialists such as surgeons, radiologists and cardiologists, emergency medical technicians, physician's assistants, nurse practitioners, nurses, nurse's aides, pharmacists, dieticians, microbiologists, and the like. The remote computers may also be physically located in non-traditional medical care environments so that the entire health care community is capable of integration on the network. Remote computers 28 may be a personal computer, server, router, a network PC, a peer device or other common network node, and may include some or all of the elements described above relative to server 22. Computer network 26 may be a local area network (LAN) and/or a wide area network (WAN), but may also include other networks. Such networking environments are commonplace in offices, enterprise-wide computer networks, intranets and the Internet. When utilized in a WAN networking environment, server 22 may include a modem or other means for establishing communications over the WAN, such as the Internet. In a networked environment, program modules or portions thereof may be stored in server 22, or database cluster 24, or on any of the remote computers 28. For example, and not limitation, various application programs may reside on the memory associated with any one or all of remote computers 28. It will be appreciated that the network connections shown are exemplary and other means of establishing a communications link between the computers may be used.

A user may enter commands and information into server 22 or convey the commands and information to the server 22 via remote computers 28 through input devices, such as keyboards, pointing devices, commonly referred to as a mouse, trackball, or touch pad. Other input devices may include a microphone, satellite dish, scanner, or the like. Server 22 and/or remote computers 28 may have any sort of display device, for instance, a monitor. In addition to a monitor, server 22 and/or computers 28 may also include other peripheral output devices, such as speakers and printers.

Although many other internal components of server 22 and computers 28 are not shown, those of ordinary skill in the art will appreciate that such components and their interconnection are well known. Accordingly, additional details concerning the internal construction of server 22 and computer 28 need not be disclosed in connection with the present invention.

The method and system of the present invention receives clinical agent information or genetic test result value, and provides information regarding the genetic association relevant to the information input and/or initiates actions within the healthcare system. Although the method and system are described as being implemented in a WINDOWS operating system operating in conjunction with a comprehensive healthcare network, one skilled in the art would recognize that the method and system can be implemented in any system supporting the receipt and processing of clinical agent information or genetic test results.

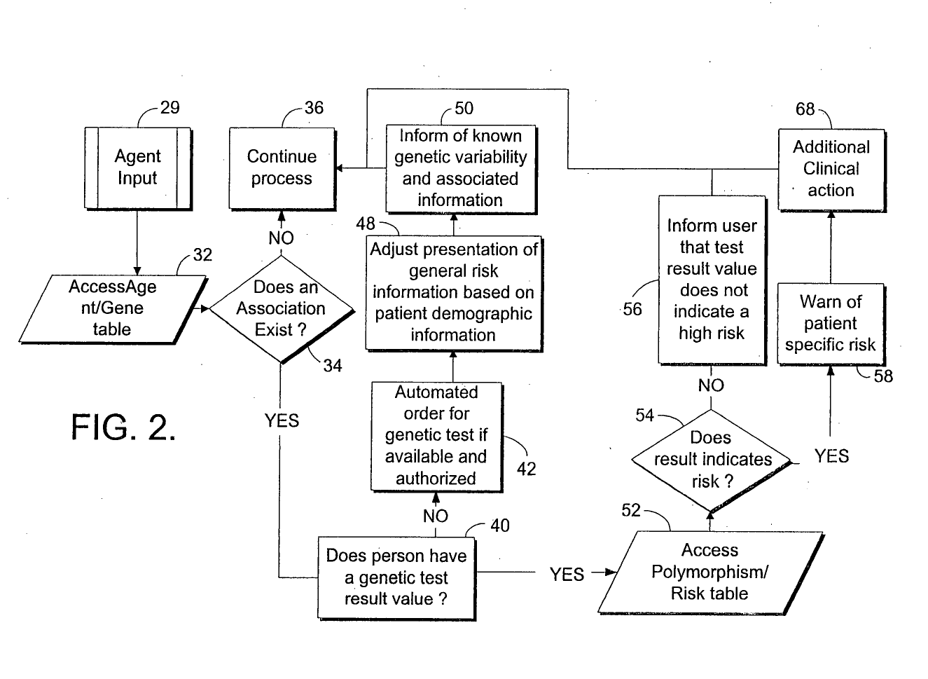

With reference to FIG. 2, in the first embodiment of the present invention, a system and method are provided for considering genetic information to determine the risk of an atypical clinical event (ACE) if a specified clinical agent is administered to the patient. A typical clinical events as used herein include adverse reactions, but also includes reactions to the clinical agent resulting in little or no benefit to the patient. Clinical agents as used herein include drugs, pharmaceuticals, nutriceuticals, foods, salves, dietary supplements and the like.

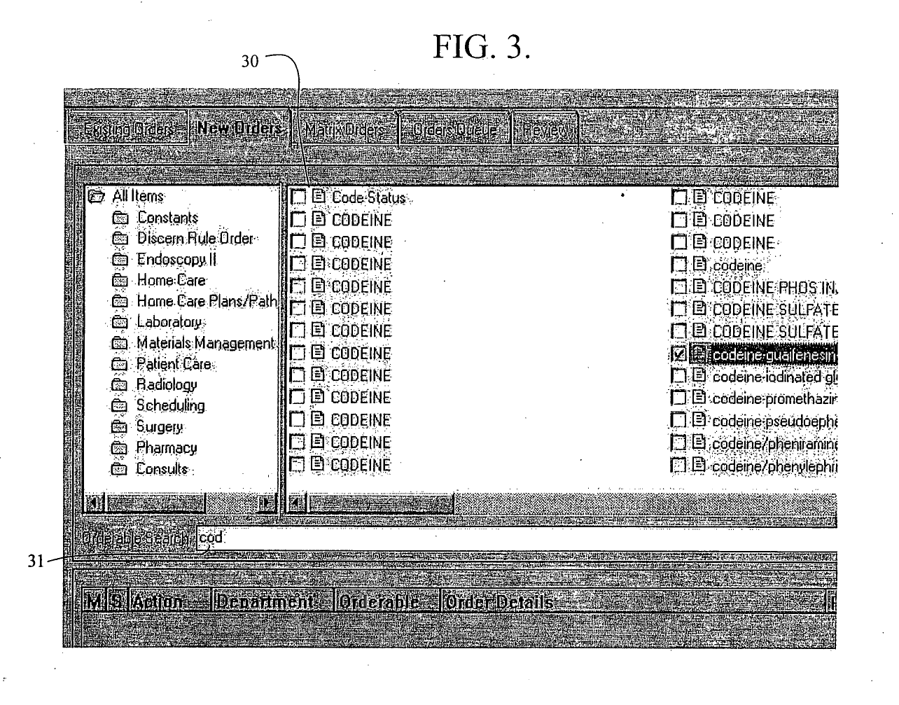

In the first step of the system, information identifying a clinical agent is input into the system at step 29. Preferably, the agent is selected at one of the remote computers 28 and transmitted to the control server 22 via the network 26. By way of example, as seen in FIG. 3, an exemplary user interface window 30 is shown. The user interface window presents a graphical user interface of the conventional kind for selecting the agent from a comprehensive list. The agent list could include the generic names as shown in FIG. 3, but may also include abbreviations, trade names, formal medical nomenclature, alternative doses for a given agent and other formats for identify the agent. For example, multiple entries for each clinical agent may be included in the list, and each entry could relate to a specific dosage or a range of dosages recommended for each agent.

The agent may be selected from the list of agents displayed on the user interface window 30 in a variety of ways. For instance, the clinician operating the system may view an expansive list of clinical agents, and select the desired agent by inputting the complete name, or by keying in a portion of the name of the desired agent at field 31 to access the relevant portion of the agent list and selecting the desired agent. Any of a number of input devices and techniques may also be utilized at this step of the method and in each of the subsequent steps wherein user input is received. For instance, another common input is from a recording made by a surgeon's dictation equipment by voice recognition techniques.

Once the clinical agent input is received, at step 32 the system accesses an agent/gene association table maintained in the memory of the system such as in the database cluster 24. Within this environment, the informational databases may be stored at any of a number of locations within the system. For instance, the agent/gene table may be accessed via a global computer network such as the Internet rather than being stored in the data cluster as described above with reference to the preferred embodiment. The table includes a list of agents and genes associated with the response to each of the agents. As appreciated by those of skill in the art, a single agent may have associations with more than one gene. Similarly, a single gene may have associations with more than one agent. An exemplary portion of an agent/gene association table is shown as Table 1:

| Agent | Gene |
|---|---|
| Codeine | CYP2D6 |
| Halothane | CYP2A6 |
| Halothane | RYR1 |
| Lidocaine | CYP3A4 |
| Terfenadine | CYP3A4 |
| Terfenadine | CYP3A5 |
| Terfenadine | CYP3A7 |
| Terfenadine | KvLQT1 |
| Mercaptopurine | TPMT |

As more information regarding agent/gene associations is learned, the table will be updated so that physicians and other operators of the system will have the most current information at their disposal. A number of variations are within the purview of the data structure exemplified in Table 1. For instance, much like the agent selection list, the data structure could accommodate input identifying the agent by an abbreviation, trade name and other formats at step 29. Likewise, other nomenclatures for identifying genes may be used, including formal medical nomenclatures and identifiers such as those used in public databases.

Next, at step 34, the system determines if an association exists between the clinical agent input and a certain gene or number of genes. Stated another way, the system determines if the products of the genes are likely to interact with the agent to result in an atypical clinical event. If an association is not present, the system continues at step 36. In a comprehensive automated healthcare system, the system would proceed without further concern regarding genetic information for the particular agent. Alternatively, the process may continue at step 36 by resetting the agent input and returning to step 29 until the next agent input is received.

If an association does exist, at step 40, the system determines if a genetic test result value is stored for the gene or genes associated with the agent. The test result value may be from any number of DNA testing techniques including DNA sequence analysis, cytogenetic testing, and Polymerase Chain Reaction (PCR) based analysis. Preferably, the system would access the patient's electronic medical record to determine if the record contained a medical test result value. Typically, patient identification information is received by the system at any of a number of steps in the method or before the method is initiated. For instance, the patient may be identified at step 29 along with the clinical agent, or may be inputted at step 40 when the patient's data becomes relevant. The method may include steps requiring authorization of the user to access the particular patient information and similar security measures known by those of skill in the art. Alternatively, rather than a patient based data structure such an electronic medical record, the data structure may be stored any of a number of manners associating a genetic test result value to the patient.

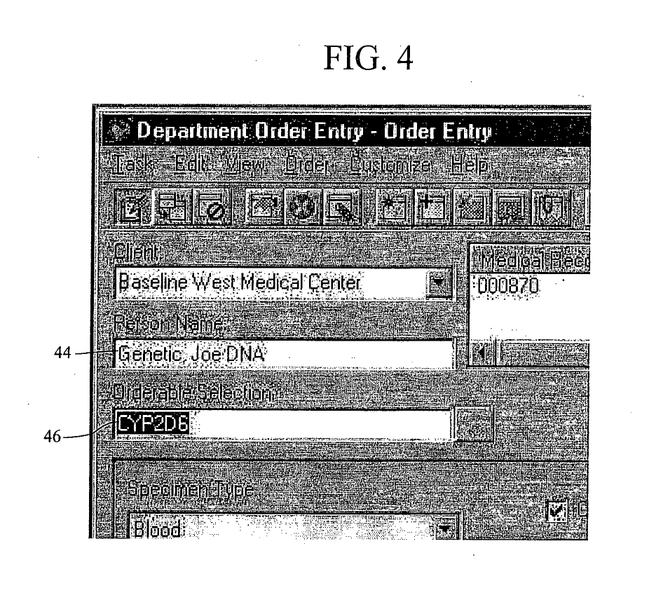

If the patient has not had a genetic test performed relevant to the genetic trait, the system may order a test at step 42 if the test is available and authorization is received. With respect to authorization, the system may either automatically order the test, or the clinician's input may be sought by the system. Whether a clinician's input is required may depend on cost of the test, the severity and likelihood of a genetic variation as determined by the system and described below or other factors. With brief reference to FIG. 4, a representative genetic test ordering window is shown. If, at step 42, the system requires clinician authorization, the system could display a window with the patient's name provided in field 44 and the orderable genetic test identified in field 46. Upon approval by the clinician, the test would be ordered and the authorization recorded on the patient's medical record.

Other clinical actions besides ordering the test may be initiated at this stage in the process. For instance, the system could produce a warning to the clinician that the agent should be suspended pending results from the genetic test. By way of an additional example, the system could request input regarding whether the patient's parents had the mutated gene in order to determine the likelihood of the existence of the gene mutation in the patient being treated. Other examples include automatically rescheduling a procedure or ordering a follow up test.

Next, at step 48, if the specific genetic test result information is not available for the patient, the system calculates the likelihood that the patient displays the genetic mutations linked with the gene or genes associated with the clinical agent. Preferably, the system accesses a database containing personal information about the patient. If personal information relevant to the calculation of genetic variability is unavailable, the system informs the user of the genetic variability and associated information relevant to the general population.

If demographic information about the patient is available, the system uses that information to adjust the display of the comments described above. As known in the art and as set forth in the example that follows, the gender, racial, ethnic, geographic distribution information are indicative of genetic predisposition to certain conditions. For instance, numerous studies have found that the frequency of mutations in drug acetylation may vary among populations of different ethnicity and geographic origin. Meyer et al., Molecular Mechanisms of Genetic Polymorphisms of Drug Metabolism, Annu. Rev. Pharmacol. Toxicol., 1997: 37: 269-295. By way of example, 40-70% of those in populations of European and North American descent are slow acetylators of izoniazid, compared to only 10-30% of those from Pacific Asian populations. Other genes have widely varying genotypic frequencies. For example, mutated forms (or alleles) of one particular gene, CYP2D6, vary greatly between Caucasian, Asian, Black African, and Ethiopian and Saudi Arabian populations. Ingelman-Sundberg et al, Polymorphic human cytochrome P450 enzymes: an opportunity for individualized drug treatment, Trends. Pharmacol. Sci., 1999: 20(8):342-349. Other traits are influenced by genes in the gender determining chromosomes, X and Y. Additionally, information regarding other genetic illnesses and the genetic characteristics of the patient's family members are also factors in determining the likelihood of genetically influenced risks, and adjusting the presentation of potential risk factors to the clinician.

The system accounts for the relevant information, and adjusts the display of the information at step 48. In the simple cases, a single demographic factor of the patient will serve as the basis for adjusting the presentation. In more complex cases, such as when other relevant factors are available, or if the patient is of multiracial descent, each of the relevant factors guide the determination and presentation of risk information. The demographic adjustments in the present system rely upon rules stored within the memory of the system. Like the gene/agent association table, these rules will develop and improve as relationships between population genetics and variations in drug response are understood.

Next, at step 50, a message is constructed informing the user of the likelihood of the genetic variability based on the rules described above at step 50. In addition to the risk information, the message may include information stored in the system regarding the severity of the atypical clinical event, the known remedies, and additional details about the molecular nature of the genetic polymorphism. Preferably, a graphical display window is generated indicating the percentage of the patient's relevant population that have the mutated gene and the affects associated with the gene. Once this message is delivered to the system, the process is continued at step 36.

If the patient does have a stored genetic test result value, a polymorphism/risk table is accessed at step 52. The polymorphism/risk table relates polymorphism information to the level of risk for a particular agent. An example of a portion of a polymorphism/risk table is shown in

TABLE 2

| | Poly- | | | |
|---|---|---|---|---|
| Gene | morphism | Agent | Phenotype | Risk |
| CYP2D6 | Duplication | Debrisoquine | Extensive | Need more |
| | | | metabolizer | frequent or |
| | | | | higher dose |
| CYP2D6 | C2850T | Debrisoquine | Poor | Non- |
| | | | metabolizer | responsive |
| CYP2D6 | G3828A | Debrisoquine | Poor | Non- |
| | | | metabolizer | responsive |
| TPMT | G460A | Mercaptopurine - | Poor | Change to |
| | | 75 mg/day | metabolizer | lower dose |
| TPMT | G460A | Mercaptopurine - | Poor | Limited risk |
| | | 10 mg/day | metabolizer | |

Like the gene/agent table, as more information regarding agent/gene associations are accepted, the table will be updated and improved. Also, values for polymorphisms not associated with risks may be incorporated in the polymorphism/risk table. Likewise, the nomenclature for the table may be widely varied without departing from the scope of the invention. Also, in one of many alternative implementations, the data from the gene/agent table and the risk/polymorphism table could be incorporated into a single data structure.

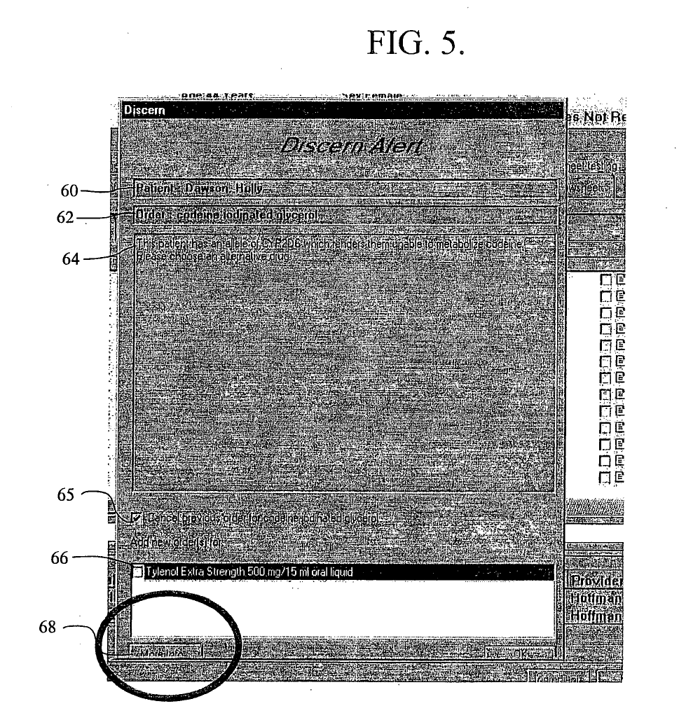

At step 54, the system determines if the specific genetic test result of the patient is indicative of a significant risk of an atypical clinical event. Preferably, the system searches the polymorphism/risk table for the medical test result value and identifies the risk associated with the result. If no significant risk is present, at step 56, the user of the system is informed that the test result does not indicate a high risk, and the process is continued at step 36. If, however, the result does indicate a risk, the user is warned of the specific risk at step 58. With brief reference to FIG. 5, a notification window is shown for exemplary purposes. In field 60, the patient's name is displayed and, in field 62, the clinical agent input at step 29 is displayed. In the main field 64, the message generated by the system is displayed warning the clinician of the patient's genetic mutation and its effect.

Next, at step 68, an additional clinical action may be taken based on the risk determined by the system. For example, the risk may be recorded in a central medical system into the patient's electronic medical record, the administration of the clinical action may be delayed or canceled, additional therapy scheduled, an alternative agent may be selected, or the patient may be referred to a clinical counselor. By way of example, with reference back to FIG. 5, the clinical action of canceling the previous order is displayed at box 65. The system is default to cancel the action absent input from the clinician to the contrary. Also, as displayed in FIG. 5, the system may display an alternative clinical agent within field 66 that is not associated with the genetic mutation of the patient.

At this step of the system, additional information regarding the association of the clinical agent and the genetic mutation may be obtained by selected the “MORE INFO” button designated at input 68. Numerous sources of information may be accessed by making this selection. For instance, the information may be embedded within the data structure stored within the system, or may be retrieved by firing an order to access information via a global computer network such as the Internet. The information may include studies about the mutation, information about alternative treatments and other materials relevant to the decision making process. Once the action is performed, the process is continued at step 36 as set forth above.

In operation, by way of a number of examples of agents having known gene associations, a number of processes are described herein. First, it is known that approximately one in three hundred people have mutations in the gene encoding thiopurine methyltransferase (TPMT) that impairs the ability to metabolize mercaptopurine (MP), a common agent used in chemotherapy treatments. Since the agent is used at near-toxic levels, patients exhibiting the mutation often die from the chemotherapy. In the present invention, a clinician such as an oncologist would input MP as a possible agent at step 29. Next, the agent/gene association table would be accessed at step 32. At step 34, the system would determine an association exists, and the system would determine if a genetic test result value for the patient was stored in the system at step 40. If a result was not stored in the system, an automated test would be ordered at step 42 without clinician authorization. Absent other patient information to adjust the display of information at step 48, the system would inform the clinician of the 0.3% mutation in the population and provide information as to the severity of the ACE at step 50. Preferably, the clinician would receive the warning visually by a similar to the window of FIG. 5, and an audible signal indicating that a warning was being delivered by the window. By way of example, the message could state that “In 0.3% of the U.S. population, mutations in the TPMT gene lead to an increased risk of cytotoxicity in response to MP.”

In a variation from this initial example, if the patient's records included information that the patient was from the Indian subcontinent, the system would consider this demographic information in determining the risk and output at step 48. It is known that only about 4 in 1000 of the Indian population is at risk of having the genetic mutation associated with the ACE. Accordingly, at step 50, the system would produce a window indicating that the risk was less for this patient than typical in the general population in the United States, or produce a substitute window information the user of the risk. By way of example, the message could state that “Four in 1000 persons from the Indian subcontinent have an increased risk of cytotoxicity in response to MP.”

Conversely, if a genetic result value was stored in the system, the polymorphism/risk table would be accessed at step 52. If the genetic test result value did not indicate that the patient has one of the mutations associated with an ACE, an output stating that the “Current test results do not indicate a high risk of this phenotype” would be provided to the clinician at step 56, an email message could be sent to the physician, or a notation made in the electronic medical record without an indication to the physician.

However, if the genetic test result indicated that the patient had a genetic mutation, the polymorphism/risk table would be accessed at step 52 and a risk indicated at step 54. For instance, the patient could have a genetic mutation in the TPMT gene in which the guanine at position 460 is replaced with adenine. When the genetic test result value for this mutation is queried within the polymorphism/risk table at step 52, the system would determine the risk of MP induced cytotoxicity, and this information would be provided to the clinician by a clear warning at step 58. Similarly, the order would be cancelled automatically at step 68, and an alternative recommendation made. Also, at step 68, the physician would be given an opportunity to approve the recommendation, and an automated order made based on the recommendation if approved by the physician.

In some cases, such as with MP therapy, the patient is unequipped to metabolize the drug in the typical dosage, but the risk of damage from the disease or condition itself has greater risks if the drug is not administered. For instance, in an exemplary case, a young patient with Acute Lymphoblastic Leukemia (ALL) may also have a severe TPMT deficiency. Typical dosages of MP of about 75 mg/m² per day would lead to intolerable toxic effects after the therapy. However, at 6% of the dosage, the toxicity would be above normal, but not at dangerous levels. Thus, in the present system, the polymorphism/risk table such as the portion displayed on Table 2, would indicate that a lower dose be prescribed at step 68.

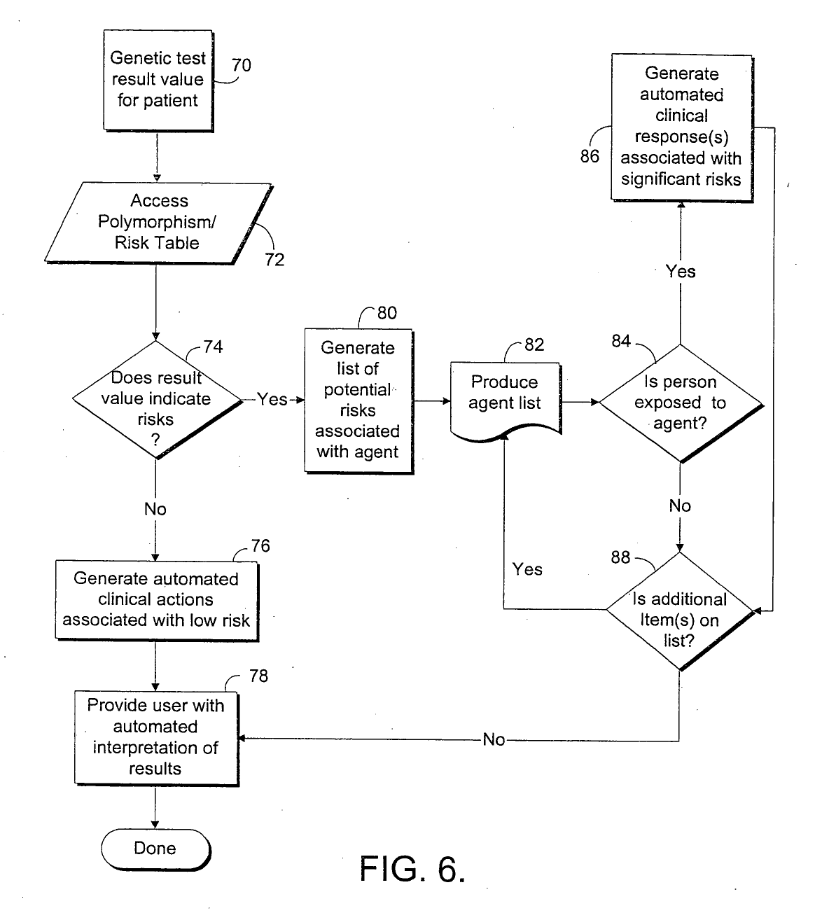

In another aspect of the invention, the system may determine the risks associated with a specific genetic test result input. With reference to FIG. 6, at step 70, a genetic test result value for a patient may be input. The genetic test result is similar to the results sought in step 40 of the embodiment of the invention described above. Next, for the specific genetic test result, the polymorphism/risk table is queried at step 72. If, at step 74, the system determines that few risks are associated with a specific genetic test result value, clinical actions associated with a low risk are generated at step 76. For example, the system could add a comment to an integrated electronic medical record that no risks were determined for the test result value. Next, at step 78, the user would be provided with interpretation of the results. In this case, the user would be provided with an indication that the genetic test result was not associated with any known risks or, specifically, clinical agents that may result in an atypical clinical reaction.

Conversely, if genetic risks are known for the specific genetic test result at step 74, a list of potential risks are generated at step 80. From this list, a list of agents that are associated with the mutation indicated by the genetic test result is generated at step 82. At step 84, for the first agent on the list, the system determines if the patient has been exposed to the agent or may prospectively be exposed to the agent. If the patient has been exposed to the agent, at step 86, the system generates an automated clinical response associated with the high risk. This response may include suspension or cancellation of the order, placing an alternative order, paging the ordering clinician, ordering follow-up tests, or scheduling counseling for the patient. Once this is complete, the system repeats the process for additional agents on the list generated at step 82. Once all of the agents are considered at step 88, the user is provided with an automated interpretation of the results at step 78. In this case, the interpretation would indicate to the user that certain clinical agents should be avoided due to the genetic predisposition to an atypical clinical reaction and other information similar to step 50 of the embodiment described above.

In operation, by way of example, a genetic test result value for the TPMT gene is input at step 70. The polymorphism/risk table is queried at step 72, and the system determines that no risk is associated with the value at step 74. Thus, at step 76, a comment could be generated about the result, and an interpretation of the medical test result added to the patient's electronic medical record at step 78.

If the genetic test result value input at step 70 had associated risks on the polymorphism/risk table at step 72, such as G460 as shown in Table 2, the system would make the association at step 74. Since more than one risk may be associated with the genetic test result value, at step 80, the system generates a list of potential risks when potential agents are administered. Once the list is produced at step 82, the system queries whether the person is exposed to the agent at step 84. If the patient does not have exposure to each successive agent on the list as determined within steps 84, 88, and 82, the system ultimately provides an interpretation of these results at step 78.

By way of example, if MP is on the agent list produced at step 82, and the system determines that the person is exposed to MP at step 84, the system generates an automated clinical response at step 86. For instance, the system could produce an urgent page to the treating physician and the attending staff to immediately inform them that MP should no longer be administered to the patient. The system would determine if additional agents required action within steps 88, 82 and 84.

Since the system may be integrated with architectures spanning the healthcare organization, the system will operate to manage the risk associated with clinical agents without creating inefficiencies. The system and method of the present invention seamlessly integrates complex genetic information and unchanging genetic information into an overall healthcare system. The system allows physicians to consider the genetic implications of prescribing any one of thousands of clinical agents and instantly have information relating to significant risk considered either automatically or manual in the clinical process. By integrating unchanging hereditary information with newfound knowledge associating this information to certain clinical agents, the system will allow the caregiver to appreciate the risks that are not readily apparent from the symptoms of the patient or associated with the particular agent.

Moreover, in the preferred embodiment, the system and method is implemented into a comprehensive automated healthcare system within the context of existing storage media and clinical processes. As mentioned above, the demographic information and individualized genetic information may be stored in an electronic medical record. Likewise, the system and method of the present invention is capable of integration with portions of the comprehensive healthcare systems dealing with conventional drug-drug interactions and allergic reactions. One such system is described in U.S. Pat. No. 5,833,599 to Robert W. Schrier et al., issued on Nov. 10, 1998, herein incorporated by reference in its entirety. For instance, when used with the system described in U.S. Pat. No. 5,833,599, the warnings relating to the risks of genetic mutation in the general population could be provided by an additional paragraph in the stored warning information.

As mentioned at the outset, consideration of the hereditary genetic information may be incorporated in the physician's standard of care as the implications of the information become widely known. Absent the system and method of the present invention, it would be burdensome and inefficient for physicians to consider this important, if otherwise unmanageable, genetic information. Since the patient's genotype does not vary throughout their lifetime, testing for most traits is only required once during the patient's life. The inclusion of this information in the electronic medical record or other permanent data structure allows physicians to make decisions based on the latest understandings of genetic information by accessing the updated databases. By raising the standard of care, and providing an incentive for genetic testing, the number of ACEs could be dramatically decreased.

The system is integrated with a comprehensive healthcare system so that the risks attributable to genetic variations are considered automatically at each location and phase of the patient care. Unlike previous systems, the system of the present invention requires little genetics training to realize the benefits of the system. Thus, caregivers in all fields of the healthcare industry may benefit from the improved understanding of the affects of genetic variability on patient care. Moreover, the system can process the genetic information and initiate clinical actions without requiring further user intervention.

The flexibility of the system provides benefits in related areas since the system is not limited by function or input type. Namely, the identified agent does not have to be administered. For instance, the system may be used by the clinician to learn more about the agent rather than as a tool for making actual patient care decisions.

Additionally, the system could be implemented for agents other than drugs and the like such as lab tests, surgical procedures, therapies, orderables, diagnoses, reflex and symptoms. For instance, the system could determine if the patient is predisposed to react adversely to a particular test. If the predisposition was identified, the physician could be warned, the test canceled, the risk documented, or any of a number of clinical actions performed.

Additionally, the manner in which the system accesses the gene-agent table and polymorphism/risk table to provide warnings to the clinicians regarding genetic information provides an effective and efficient structure for managing other types of genetic data. This aspect of the invention may be implemented to process genetic information outside of the patient's preexisting and unchanging genetic traits. As a first example, certain somatic mutations accumulate after one is born. Some of these somatic mutations, such as those in the p53 gene, predispose to risk of cancer. While detection of these mutations requires periodic testing, the information management structures of the present invention, namely the agent/gene tables and polymorphism/risk tables could be used to manage this type of data. In another example, it is well documented that the genome of the HIV-1 virus mutates and develops resistance to known drug treatments. Simple systems have been implemented to test periodically to determine the genotype of the virus to assess the resistance based on the genotype of the gene and the resistance actually manifested. These systems are similar to previous drug allergy systems, and are not particularly adept in handling complex genetic information. Nor are they integrated into a full clinical record. By using the data structures of the present system, genetic information besides that of the patient may be processed more efficiently than in these systems. Likewise, other exogenous sources of DNA such as other viruses, bacteria, and other genes that are present in the patient such as genes injected into patient's body in gene therapy treatment currently under development can be used to drive similar rules.

Although the invention has been described with reference to the preferred embodiment illustrated in the attached drawing figures, it is noted that substitutions may be made and equivalents employed herein without departing form the scope of the invention as recited in the claims. For example, additional steps may be added and steps omitted without departing from the scope of the invention.

The invention claimed is:

1. A method in a computer system for preventing atypical clinical events related to information identified by DNA testing a person, comprising the steps of:
receiving clinical agent information, the clinical agent information including an identifier of the agent;
determining if a gene is associated with the clinical agent information, and if so, obtaining a genetic test result value for the associated gene of the person;
comparing the genetic test result value to a list of polymorphism values associated with an atypical clinical event, and
determining whether the genetic test result value correlates to a polymorphism value on the list, and if so, outputting information about the atypical clinical event associated with the polymorphism value.

2. The method of claim 1, wherein the clinical agent information includes a dosage of the identified clinical agent.

3. The method of claim 1, wherein the clinical agent information is received over a communication network from a remote computer.

4. The method of claim 1, wherein the step of determining if a gene is associated with the clinical agent information includes querying a first data structure containing agent-gene associations and determining if a gene has one or more variants associated with an atypical response to the identified clinical agent.

5. The method of claim 4, wherein a plurality of genes have one or more variants associated with an atypical response to the identified clinical agent.

6. The method of claim 4, further comprising the step of initiating a clinical action if a gene has at least one variant associated with an atypical response to the identified clinical agent.

7. The method of claim 6, wherein the clinical action is providing a warning that the identified agent should not be administered.

8. The method of claim 6, wherein the clinical action is ordering a genetic test for the person.

9. The method of claim 6, wherein the clinical action is canceling another clinical action.

10. The method of claim 1, wherein the genetic test result value is obtained from an electronic medical record of the person stored within a comprehensive healthcare system.

11. The method of claim 1, wherein the step of comparing includes querying a second data structure containing polymorphism-atypical result associations.

12. The method of claim 1, wherein the second data structure includes information about risks associated with the atypical clinical event.

13. The method of claim 12, wherein the step of outputting information includes accessing the risk information in the second data structure.

14. The method of claim 1, wherein the step of determining if a gene is associated with the clinical agent information includes querying a first data structure containing agent-gene associations and wherein the step of comparing includes querying a second data structure containing polymorphism-atypical result associations, wherein the first data structure and second data structure are integrated as a single data structure.

15. The method of claim 1, wherein the output information includes a message containing a warning of the patient specific risk.

16. The method of claim 1, wherein the clinical agent information includes a dosage of the identified clinical agent, and wherein the second data structure includes information about risks associated with various dosages of the identified clinical agent.

17. The method of claim 1, further comprising the step of outputting information that the person is not at risk if the genetic test result value does not correlate to a polymorphism value.

18. A computer system for preventing atypical clinical events related to information identified by DNA testing a person, comprising:
a receiving component that receives clinical agent information, the clinical agent information including an identifier of the agent;
a first determining component that determines if a gene is associated with the clinical agent information;
an obtaining component for obtaining a genetic test result value for the associated gene of the person;
a comparing component for comparing the genetic test result value to a list of polymorphism values associated with an atypical clinical event;
a second determining component that determines whether the genetic test result value correlates to a polymorphism value on the list, and
an outputting component that outputs information about the atypical clinical event associated with the polymorphism value.

19. The computer system of claim 18, wherein the clinical agent information includes a dosage of the identified clinical agent.

20. The computer system of claim 18, wherein the clinical agent information is received over a communication network from a remote computer.

21. The computer system of claim 18, wherein the first determining component includes a querying component that queries a first data structure containing agent-gene associations, and wherein the system further comprises a third determining component that determines if a gene has one or more variants associated with an atypical response to the identified clinical agent.

22. The computer system of claim 21, wherein a plurality of genes have one or more variants associated with an atypical response to the identified clinical agent.

23. The computer system of claim 21, further comprising an initiating component that initiates a clinical action if a gene has at least one variant associated with an atypical response to the identified clinical agent.

24. The computer system of claim 23, wherein the clinical action is providing a warning that the identified agent should not be administered.

25. The computer system of claim 23, wherein the clinical action is ordering a genetic test for the person.

26. The computer system of claim 23, wherein the clinical action is canceling another clinical action.

27. The computer system of claim 18, wherein the genetic test result value is obtained from an electronic medical record of the person stored within a comprehensive healthcare system.

28. The computer system of claim 18, wherein the comparing component includes a querying component that queries a second data structure containing polymorphism-atypical result associations.

29. The computer system of claim 18, wherein the second data structure includes information about risks associated with the atypical clinical event.

30. The computer system of claim 29, wherein the outputting component includes an accessing component that accesses the risk information in the second data structure.

31. The computer system of claim 18, wherein the first determining component includes a querying component that queries a first data structure containing agent-gene associations and wherein the comparing component includes a second querying component that queries the second data structure containing polymorphism-atypical result associations, wherein the first data structure and second data structure are integrated as a single data structure.

32. The computer system of claim 18, wherein the output information includes a message containing a warning of the patient specific risk.

33. The computer system of claim 18, wherein the clinical agent information includes a dosage of the identified clinical agent, and wherein the second data structure includes information about risks associated with various dosages of the identified clinical agent.

34. The computer system of claim 18, further comprising a second outputting component that outputs information that the person is not at risk if the genetic test result value does not correlate to a polymorphism value.

35. A computer-readable medium containing instructions for controlling a computer system for preventing atypical clinical events related to information identified by DNA testing a person, by:
receiving clinical agent information, the clinical agent information including an identifier of the agent;
determining if a gene is associated with the clinical agent information, and if so, obtaining a genetic test result value for the associated gene of the person;
comparing the genetic test result value to a list of polymorphism values associated with an atypical clinical event, and
determining whether the genetic test result value correlates to a polymorphism value on the list, and if so, outputting information about the atypical clinical event associated with the polymorphism value.

36. The computer-readable medium of claim 35, wherein the clinical agent information includes a dosage of the identified clinical agent.

37. The computer-readable medium of claim 35, wherein the clinical agent information is received over a communication network from a remote computer.

38. The computer-readable medium of claim 35, wherein the step of determining if a gene is associated with the clinical agent information includes querying a first data structure containing agent-gene associations and determining if a gene has one or more variants associated with an atypical response to the identified clinical agent.

39. The computer-readable medium of claim 38, wherein a plurality of genes have one or more variants associated with an atypical response to the identified clinical agent.

40. The computer-readable medium of claim 38, further comprising the step of initiating a clinical action if a gene has at least one variant associated with an atypical response to the identified clinical agent information.

41. The computer-readable medium of claim 40, wherein the clinical action is providing a warning that the identified agent should not be administered.

42. The computer-readable medium of claim 40, wherein the clinical action is ordering a genetic test for the person.

43. The computer-readable medium of claim 40, wherein the clinical action is canceling another clinical action.

44. The computer-readable medium of claim 35, wherein the genetic test result value is obtained from an electronic medical record of the person stored within a comprehensive healthcare system.

45. The computer-readable medium of claim 35, wherein the step of comparing includes querying a second data structure containing polymorphism-atypical result associations.

46. The computer-readable medium of claim 35, wherein the second data structure includes information about risks associated with the atypical clinical event.

47. The computer-readable medium of claim 46, wherein the step of outputting information includes accessing the risk information in the second data structure.

48. The computer-readable medium of claim 35, wherein the step of determining if a gene is associated with the clinical agent information includes querying a first data structure containing agent-gene associations and wherein the step of comparing includes querying a second data structure containing polymorphism-atypical result associations, wherein the first data structure and second data structure are integrated as a single data structure.

49. The computer-readable medium of claim 35, wherein the output information includes a message containing a warning of the patient specific risk.

50. The computer-readable medium of claim 35, wherein the clinical agent information includes a dosage of the identified clinical agent, and wherein the second data structure includes information about risks associated with various dosages of the identified clinical agent.

51. The computer-readable medium of claim 35, further comprising the step of outputting information that the person is not at risk if the genetic test result value does not correlate to a polymorphism value.
